(19) United States
(12) Patent Application Publication
(10) Pub. No.: US 2026/0007797 A1
(43) Pub. Date: Jan. 8, 2026

(54) ATOMIZING ASSEMBLY WITH AIR INLET TUBE AND FRAGRANCE DIFFUSER

(71) Applicant: AROMA PERFECTO LLC, Diamond Bar, CA (US)

(72) Inventor: Yong WU, Guangzhou (CN)

(21) Appl. No.: 19/019,565

(22) Filed: Jan. 14, 2025

(30) Foreign Application Priority Data
Jul. 2, 2024 (CN) 202421552718.7

(51) Int. Cl. A61L 9/14; A61L 9/12

(52) U.S. Cl. CPC A61L 9/14; A61L 9/122; A61L 2209/134

(57) ABSTRACT

An atomizing assembly includes a container and an atomizer including a cover body and an atomizing core. The cover body is provided with at least one through hole and the atomizer is correspondingly provided with at least one air inlet tube having a certain length in communication with the respective through hole, so as to allow external air to enter the atomizer through the air inlet tube.

### CROSS REFERENCE TO RELATED APPLICATIONS

The present application claims priority from Chinese Application No. CN 202421552718.7 filed on Jul. 2, 2024, all of which are hereby incorporated herein by reference.

### TECHNICAL FIELD

The present disclosure relates to the technical field of spray devices, particularly to an atomizing assembly with an air inlet tube and a fragrance diffuser.

### BACKGROUND

With diversification of people's lifestyles, atomizers for adjusting indoor air and fragrance diffusers equipped with atomizers are increasingly available in people's daily lives. As shown in FIG. 1 and FIG. 2, the main structure of an atomizer generally includes a container A for storing liquid to be atomized, a cover body B that is sealed connection with the opening of the container A, and an atomizing core C installed inside the cover body B. The atomizing core C internally has a liquid flow passage and an air flow channel, with the outlet of the liquid flow passage and the outlet of the air flow passage being positioned proximate to each other at a certain angle. Regarding the connection of atomizing core C, the liquid flow passage of the atomizing core C is connected to a liquid suction tube D, which extends to the bottom of the internal cavity of the container A, while the air flow passage of the atomizing core C is connected externally to a high-pressure generation device for producing high-speed airflow, such as an air pump, through a pipe. When the air pump operates, the high-speed airflow is ejected from the outlet of the air flow passage to create negative pressure at the outlet of the liquid flow passage when passing over the outlet of the liquid flow passage. Under the effect of this negative pressure, liquid from the interior of the container A can enter the liquid flow passage of the atomizing core C through the liquid suction tube D and flow out from the outlet of the liquid flow passage. The liquid flowing out from the outlet of the liquid flow passage then makes contact with the high-speed airflow ejected from the air flow passage. The continuously ejected high-speed airflow impacts the liquid flowing out from the liquid flow passage to atomize the liquid and thus form atomized gas. The atomized gas formed is further discharged externally through the spray tube E at the top of cover body B along with the airflow.

However, the above-structured atomizer has consistently faced the issue of low spray volume in practical use. This issue primarily arises by insufficient air volume inside the atomizer due to the high-speed airflow ejected from the atomizing core C rapidly flowing out simultaneously during the atomizer generating the atomized gas.

To solve this problem, CN 208711985 U discloses a spray device which provides with air circulation holes in the cover body at the top of the atomizer that is in communication with the outside. This allows external air to naturally flow into the cover body at the top of the atomizer to supplement the internal air of the atomizer, thereby enhancing atomization efficiency and thus increasing the spray volume. Additionally, CN 206151930 U discloses a fragrance diffusion device, which is provided with two symmetrically positioned strip-shaped through holes in the atomizing cap body at the top of the atomizer to allow external air to naturally enter the interior of the atomizer therethrough, ensuring sufficient air volume inside the atomizer and thereby improving atomization efficiency and further increasing spray volume.

Although the prior art above can increase the spray volume of the atomizer, such designs introduce new challenges. In practical use, due to through holes directly formed in the cover body of the atomizer, noise generated by the atomizing core inside the cover body when ejecting high-speed airflow would be directly transmitted externally through such through holes in the cover body. This causes the atomizer and fragrance diffuser to produce noticeable noise during operation, which not only affects user experience, but also poses risks of liquid leakage, especially when the atomizer is tilted, liquid inside the container may be prone to leaking through the through holes in the cover body.

### SUMMARY

To address the above-mentioned technical problems existing in the prior art, the present disclosure provides an atomizing assembly with an air inlet tube and a fragrance diffuser, which can improve atomization efficiency and increase atomization volume, as well as effectively reduce noise during operation and can, to some extent, prevent liquid leakage from the atomizing assembly.

One aspect of the present disclosure provides an atomizing assembly with an air inlet tube, including an atomizer and a container. The atomizer includes a cover body and an atomizing core with an air outlet. The cover body is provided with at least one through hole, and the atomizer is correspondingly provided with at least one air inlet tube having a certain length, which is in air communication with the respective through hole to allow external air to enter the interior of the atomizer through the air inlet tube.

In particular, the end of the at least one air inlet tube close to the atomizing core has an air outlet of the air inlet tube, such air outlet is preferably located in a height position inside the cover body lower than the air outlet of the atomizing core or higher than the air outlet of the atomizing core no more than 20 mm.

According to a preferable embodiment, the at least one air inlet tube vertically extends vertically inside the cover body toward the interior of the container, name extends along the length of the container.

According to a preferable embodiment, the atomizing assembly is preferably provided with a plurality of through holes and air inlet tubes, with the number of the through holes being less than or equal to 8, and each through hole is in air communication with a respective air inlet tube. The air inlet tubes are preferably distributed at different positions inside the cover body.

According to a preferable embodiment, the air inlet tube is in form of a circular tube or elliptical tube or polygonal tube.

In particular, the cross-sectional area of the air inlet tube is preferably between 0.15 mm² and 15 mm².

According to a preferable embodiment, the extension length of the air inlet tube inside the cover body is from 5 mm to 200 mm.

According to a preferable embodiment, the cover body is provided with a spray tube disposed above the atomizing core, which is configured to allow the atomized gas ejected by the atomizing core to enter the spray tube upward and be discharged externally.

According to a preferable embodiment, the inner diameter of the spray tube is from 1 mm to 10 mm. The at least one air inlet tube has a cross-sectional area between 0.15 mm² and 15 mm².

Additionally or alternatively, the air inlet tube extends outside the cover body toward the exterior of the container. A baffle may be provided below the cover body, with the through hole and the air outlet of the air inlet tube located at the outer side of the baffle and the air outlet of the atomizing core located at the inner side of the baffle.

The atomizing assembly may be further provided with an airflow driving element, with the exhaust end thereof connected to the through hole at the exterior of the cover body to actively drive air into the at least one air inlet tube. The airflow driving element may include an air pump or fan.

Another aspect of the disclosure provides a fragrance diffuser, including a housing, a high-pressure generation device for providing high-pressure airflow, and an atomizing assembly provided in the housing for producing atomized gas under the high-pressure airflow.

The atomizing assembly includes an atomizer and a container. The atomizer includes a cover body and an atomizing core with an air outlet. The cover body is provided with at least one through hole, and the atomizer is correspondingly provided with at least one air inlet tube having a certain length, which is in air communication with the respective through hole to allow external air to enter the interior of the atomizer through the air inlet tube.

The atomizing assembly of the fragrance diffuser may be the atomizing assembly mentioned above in any case.

The atomizing assembly and fragrance diffuser in the present disclosure are not only provided with at least one through hole at the cover body of the atomizer but also provided with at least one air inlet tube in communicates with respective through holes. In such way, during operation of the atomizer, external air can enter interior of the atomizer through the air inlet tube to supplement air inside the atomizer in real time, thus achieving improved atomization efficiency and increased atomization volume. Additionally, since the air inlet tube has a certain length, compared to the traditional method of directly opening through holes in the cover body, the formation of an elongated narrow tube space contributes to restricting the outward transmission of noise from inside of the cover body. Specifically, the noise generated during atomization must travel through the narrow tube space of the predetermined length of the air inlet tube before transmitting outward, thereby restricting internal noise inside the cover body from transmitting through the through holes and thus effectively reducing the external noise produced during operation of the atomizer. Furthermore, when the atomizer is tilted, the elongated air inlet tube can further prevent liquid inside the container from directly leaking out through the through hole within the cover body, helping to prevent liquid leakage from the atomizer to some extent.

### BRIEF DESCRIPTION OF THE DRAWINGS

To more clearly illustrate the technical solutions in the present disclosure or prior art, the drawings needed in the description of embodiments or prior art will be briefly introduced below. Obviously, the drawings described below represent only some embodiments of the present disclosure not limited to the present invention:

FIG. 1 is an external structural view of an atomizer in prior art;

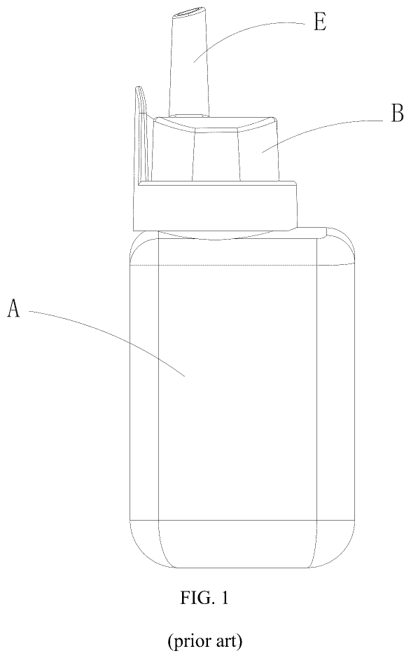

FIG. 2 is an internal structural view of an atomizer in prior art;

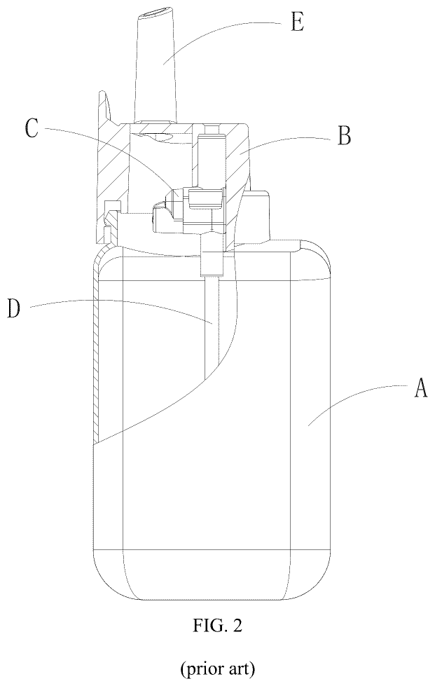

FIG. 3 is an external structural view of an atomizing assembly according to an embodiment of the present disclosure;

FIG. 4 is a partial structural view of FIG. 3, with a cover body omitted;

FIG. 5 is an internal structural view of the cover body of the present disclosure;

FIG. 6 is an internal structural view of an atomizing assembly according to an embodiment of the present disclosure;

FIG. 7 is a partial structural view of FIG. 6, with a container omitted;

FIG. 8 is a perspective view of an atomizing assembly according to an embodiment of the present disclosure, in which the cover body is provided with a plurality of through holes and air inlet tubes;

FIG. 9 is another perspective view of the atomizing assembly according to an embodiment of the present disclosure, in which the cover body is provided with a plurality of through holes and air inlet tubes;

FIG. 10 is a partial structural view of an atomizing assembly according to an embodiment of the present disclosure, in which an air inlet tube extends outside a cover body, with an air outlet of the air inlet tube located in a height position inside the cover body higher than an air outlet of the atomizing core;

FIG. 11 is a partial structural view of the cover body in FIG. 10;

FIG. 12 is a partial structural view of an atomizing assembly according to an embodiment of the present disclosure, in which an air inlet tube extends both outside and inside a cover body, with an air outlet of the air inlet tube located in a height position inside the cover body lower than an air outlet of the atomizing core;

FIG. 13 is a schematic view of a fragrance diffuser.

Reference signs: 1 container, 2 atomizing core, 21 air outlet of the atomizing core, 3 cover body, 31 spray tube, 32 baffle, 4 air inlet tube, 41 air outlet of the air inlet tube, 5 through hole, 6 mounting hole of an external high-pressure generation device, 7 airflow driving element, 100 housing, 200 high-pressure generation device, 300 atomizing assembly.

### DETAILED DESCRIPTION OF THE EMBODIMENTS

The embodiments of the present disclosure will be described in detail below, with examples of these embodiments shown in the accompanying drawings. Throughout the description, the same or similar reference numerals represent the same or similar elements or elements with the same or similar functions. The embodiments described below with reference to the accompanying drawings are exemplary and are provided solely for the purpose of illustrating the present disclosure and should not be construed as limiting the scope of the present disclosure. It should be understood that the directional terms “center,” “longitudinal,” “transverse,” “length,” “width,” “thickness,” “upper,” “lower,” “front,” “rear,” “left,” “right,” “vertical,” “horizontal,” “top,” “bottom,” “inner,” “outer,” “clockwise,” “counterclockwise,” “axial,” “radial,” “circumferential,” etc., used in the present disclosure's description indicate orientations or positional relationships based on the orientations or positional relationships shown in the drawings, and are used solely to facilitate description of the present disclosure and simplify the description. They do not indicate or imply that the referenced device or element must have a particular orientation or be constructed and operated in a particular orientation, and therefore should not be construed as limiting the present disclosure.

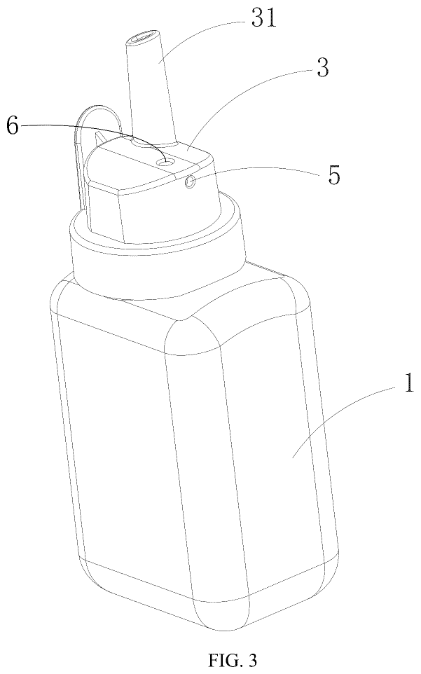

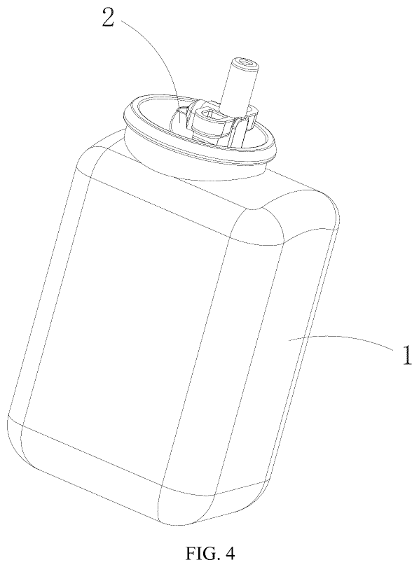

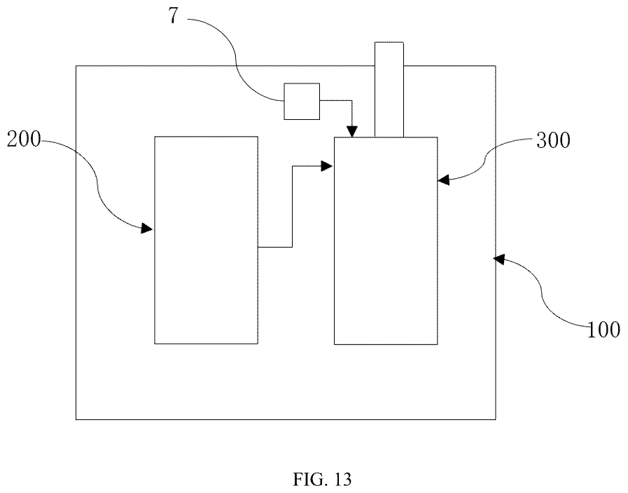

FIG. 3 and FIG. 4 are provided with an atomizing assembly having an air inlet tube, including an atomizer and a container 1. The container 1 is configured to store liquid to be atomized for forming atomized gas. The atomizer includes a cover body 3 and an atomizing core 2. The atomizing core 2 is installed at the opening at the top of container 1, and the cover body 3 is installed at the opening at the top of container 1. The cover body 3 and container 1 are connected by threads or in any other known way to seal the cover body 3 with the opening of container 1. The atomizing core 2 is accommodated in the cover body 3. The cover body 3 is provided with a mounting hole 6 on the outer side thereof, which is configured to connect an inlet tube of an external high-pressure generation device 200 (shown in FIG. 13), such as an air pump, for providing high-pressure airflow for the atomizer. In this way, the inlet tube of the air pump can be inserted through the mounting hole 6 into the cover body 3 and in communication with the air flow passage (now shown) of the atomizing core 2. The atomizing core 2 has an air outlet 21 for ejecting the high-pressure airflow to the outlet of the liquid flow passage (not shown). The cover body 3 has an integrally formed spray tube 31 positioned above the atomizing core 2, so that the atomized gas ejected by the atomizing core 2 can be moved upward and entered the spray tube 31, then finally discharged externally. In addition, the outer wall of the cover body 3 is provided with at least one through hole 5, and the atomizer is correspondingly provided with at least one air inlet tube 4 that extends a certain length towards inside of the cover body 3 and is in air communication with the respective through hole 5, thereby allowing external air to enter the atomizer through the air inlet tube 4. In this embodiment, only one air inlet tube 4 and one through hole 5 are provided.

According to the present embodiment, in addition to the through hole 5 arranged in the outer wall of the atomizer's cover body 3, an air inlet tube 4 in communication with the through hole 5 inside the cover body 3 is further provided. With such configuration, when the atomizer starts to operate, external air can enter the atomizer through the air inlet tube 4 to supplement air inside the atomizer in real time, thus improving atomization efficiency and accordingly increasing atomization volume. Additionally, since the air inlet tube 4 inwardly extends a certain length, the noise generated by the atomizing core 2 ejecting high-speed airflow can be largely blocked by the outer wall of the elongated air inlet tube 4, with only a small portion of the noise transmitting outward through the air inlet tube 4. This thus effectively reduces the noise produced during operation of the atomizer. Furthermore, when the atomizer is tilted, the elongated air inlet tube 4 can further prevent liquid inside container 1 from directly leaking out through the through hole 5 within the cover body 3, which can avoid easy liquid leakage of the atomizer to some extent.

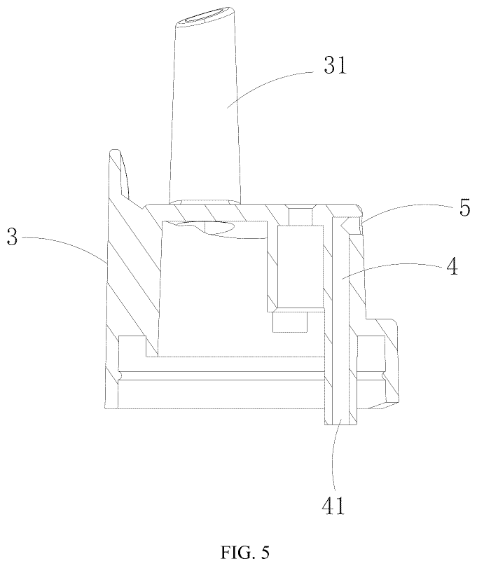

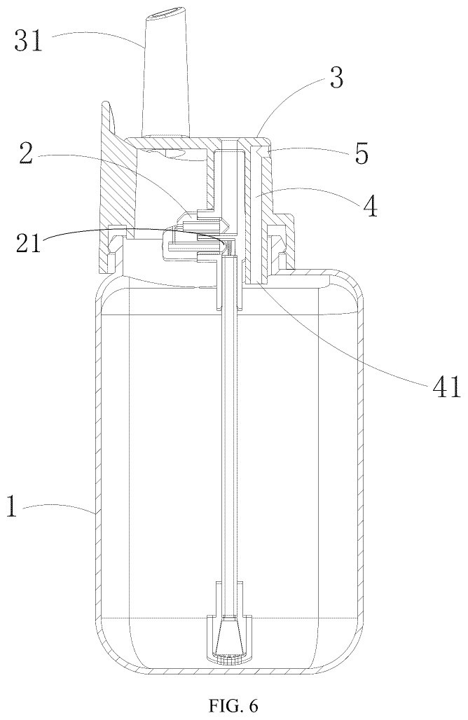

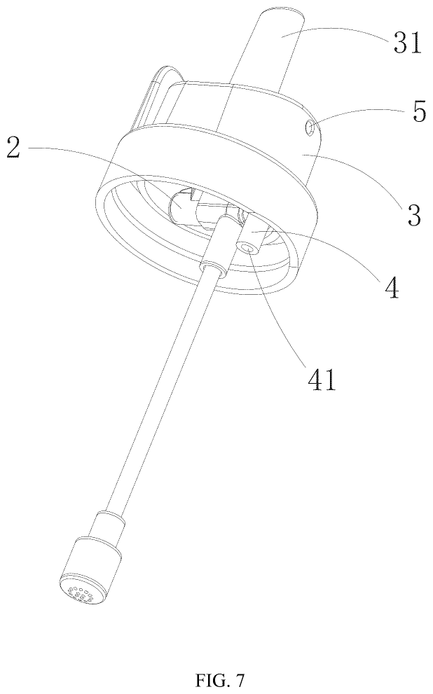

In one embodiment, as shown in FIGS. 5 to 7, the air inlet tube 4 extends vertically inside the cover body 3 toward the length of the container 1. Such configuration facilitates integral molding of the air inlet tube 4 with the cover body 3 during injection molding manufacturing. Alternatively, the air inlet tube 4 can be detachably installed inside the cover body 3. However, it is conceivable that the air inlet tube 4 can be also in form of a bend structure according to other embodiments. With such bend structure of the air inlet tube 4, liquid leakage from the atomizer can be further avoided.

In the present embodiment, the air outlet 41 at the lower end of the air inlet tube 4, i.e., the tube opening away from the through hole 5, is positioned at a height lower than the air outlet 21 of the atomizing core 2. Such configuration can prevent the high-speed airflow and atomized gas in the spray area of atomizing core 2 from entering the air inlet tube 4 through the air outlet 41 thereof. This further prevents the atomized gas from being discharged externally through the air inlet tube 4, thereby ensuring that external air can be properly supplemented into the interior of the atomizer through the air inlet tube 4.

However, in practical applications, the spray range produced by the air outlet 21 of the atomizing core 2 in atomizer products is generally sector-shaped. It is thus not absolutely necessary to position the air outlet 41 of the air inlet tube 4 lower than the air outlet 21 of the atomizing core 2. Therefore, the air outlet 41 of the air inlet tube 4 at the lower end thereof can be optionally positioned inside the cover body 3 at a height no more than 20 mm above the air outlet 21 of the atomizing core 2. This arrangement can also ensure that the high-speed airflow from the air outlet 21 of atomizing core 2 and the atomized gas do not enter the air inlet tube 4 through the air outlet 41 thereof.

The air inlet tube 4 according to the present embodiment is designed in form of a circular tube. Theoretically, the smaller the inner diameter of the air inlet tube 4 is, the less noise would be transmitted externally during atomizer operation. However, an excessively small inner diameter would affect the amount of the air entering the air tube 4. Therefore, the inner diameter of the air inlet tube 4 in the present embodiment is preferably in a range between 0.5 mm and 4 mm. Within this range of the air inlet tube 4, the transmission of noise caused during operation of the atomizer can be effectively reduced while maintaining sufficient air intake volume. Of course, the air inlet tube 4 can also be designed as an elliptical tube or polygonal tube according to other practical requirements, with an area of the tube hole equal to that of the circular tube aforementioned.

In addition, theoretically, the longer the air inlet tube 4 is, the less noise would be transmitted externally during operation of the atomizer. Accordingly, the extension length of the air inlet tube 4 in the present embodiment is preferably in a range between 5 mm and 200 mm. Similarly, theoretically, the smaller the inner diameter of the spray tube 31 is, the less noise would be transmitted externally during operation of the atomizer. However, an excessively small inner diameter would affect atomizing efficiency. Therefore, the internal diameter of the spray tube 31 in the present embodiment is preferably between 1 mm and 10 mm.

The air inlet tube 4 preferably has a cross-sectional area between 0.15 mm² and 15 mm².

In other embodiments, the spray tube 31 can be replaced by a spray opening.

An airflow driving element 7 (shown in FIG. 13) for driving air flow can be further provided according to a preferable embodiment, which can preferably include an air pump or a fan. The exhaust end of the air pump or the fan is connected to the through hole 5 at the exterior of the cover body 3 to actively drive air into the air inlet tube 4, thereby further increasing the air intake volume to the interior of the atomizer.

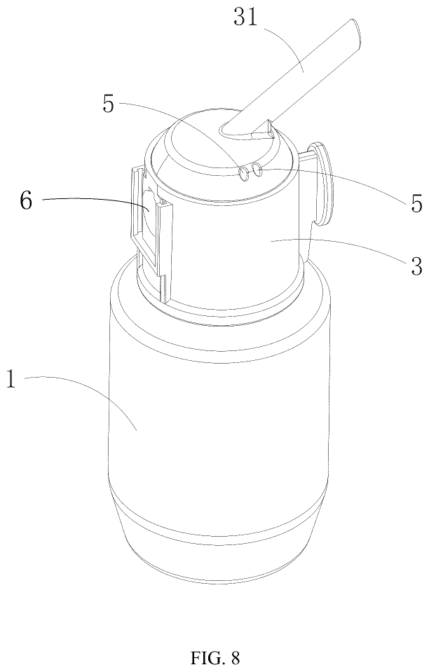

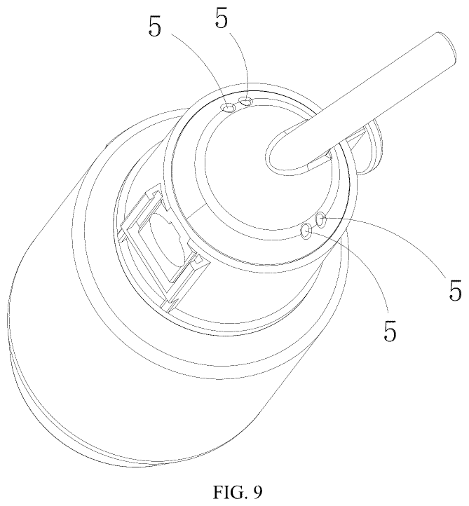

FIG. 8 and FIG. 9 show another embodiment according to the present invention. The cover body 3 of the atomizer in this embodiment is provided with a plurality of through holes 5, with the number of the through holes 5 preferably being less than or equal to 8. Correspondingly, a plurality of air inlet tubes 4 are provided, with each air inlet tube 4 inside the cover body 3 connecting to the respective through holes 5. The air inlet tubes 4 are distributed at different positions inside of the cover body 3, thus further enhancing efficiency of the external air entering the interior of the atomizer and ensuring sufficient air inside the atomizer during the spraying process, thereby in turn increasing atomization volume of the atomizing assembly.

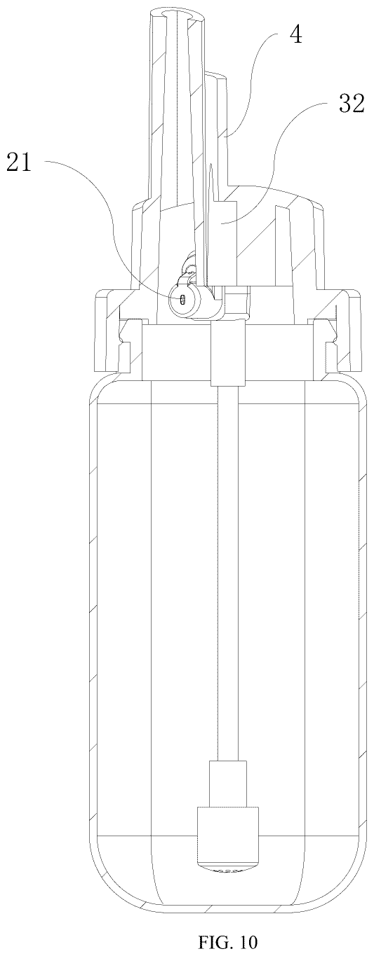

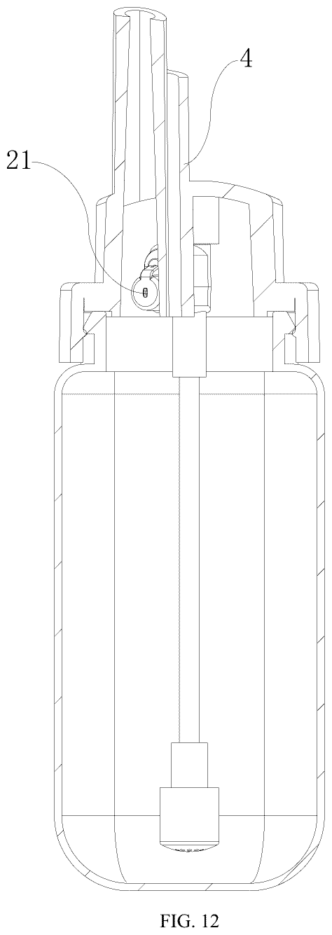

In other embodiments, as shown in FIG. 10 and FIG. 12, in addition to extending inside the cover body 3, the air inlet tube 4 can also extend outside the cover body 3 by a certain length. The air inlet tube 4 in FIG. 10 only extends outside the cover body 3, while the air inlet tube 4 in FIG. 12 extends both outside and inside the cover body 3.

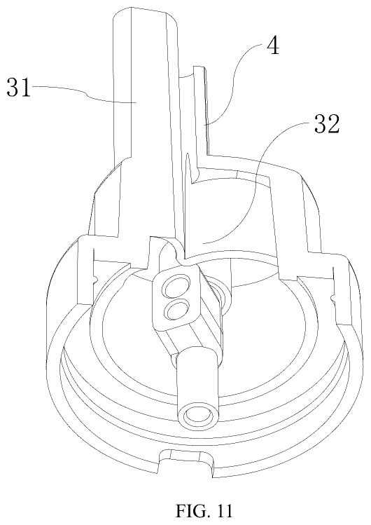

FIG. 11 shows the inner structure of the cover body 3 in FIG. 10. In this embodiment, the air inlet tube 4 only extends outside the cover body 3. The air outlet 41 of the air inlet tube 4 in this embodiment is located in a height position inside the cover body 3 higher than the air outlet 21 of the atomizing core 2. A baffle 32 is further provided below the cover body 3, with the through hole 5 and the air outlet 41 of the air inlet tube 4 located at the outer side of the baffle 32 and the air outlet 21 of the atomizing core 2 located at the inner side of the baffle 32. The design of the baffle 32 separates the air outlet 21 of the atomizing core 2 and the through hole 5 as well as the air outlet 41 of the air inlet tube 4, so that the high-speed airflow from the air outlet 21 of atomizing core 2 and the atomized gas are prevented from being discharged externally through the air inlet tube 4, even if the air outlet 41 of the air inlet tube 4 is located in a height position higher than the air outlet 21 of the atomizing core 2, thereby ensuring that external air can be properly supplemented into the interior of the atomizer through the air inlet tube 4.

It should be noted that the baffle 32 can also be adapted to the embodiment that the air inlet tube 4 extends only inside the cover body 3 and the embodiment that the air inlet tube 4 simultaneously extends inside and outside the cover body 3.

FIG. 13 shows a schematic view of a fragrance diffuser according to an embodiment of the present invention. The fragrance diffuser includes a housing 100, a high-pressure generation device 200 for providing high pressure airflow, and the aforementioned atomizing assembly 300 in any case, which is configured to produce atomized gas under the high-pressure airflow produced by the high-pressure generation device 200.

With the atomizing assembly 300 mentioned above in any case, the fragrance diffuser can effectively improve atomization efficiency and increase atomization volume, resulting in greater volume of the atomized gas with fragrance elements, meanwhile effectively reducing noise of the fragrance diffuser during operation and preventing liquid leakage from the fragrance diffuser to some extent.

Finally, it should be noted that the above embodiments are provided solely to illustrate the technical solutions of the present disclosure and are not intended to limit them. Although the present disclosure has been described in detail with reference to the above embodiments, those skilled in the art should understand that modifications can still be made to the technical solutions described in the above embodiments, or some of the technical features may be replaced with equivalents. Such modifications or replacements do not alter the essence of the corresponding technical solutions, nor do they depart from the spirit and scope of the technical solutions as disclosed in the embodiments of the present disclosure.

What is claimed is:

1. An atomizing assembly with an air inlet tube, comprising:
a container for containing liquid to be atomized; and
an atomizer, which comprises a cover body and an atomizing core having an air outlet,
wherein the cover body is provided with at least one through hole, and the atomizer is correspondingly provided with at least one air inlet tube having a certain length in communication with the respective through hole, so as to allow external air to enter the atomizer through the at least one air inlet tube.

2. The atomizing assembly according to claim 1, wherein an end of the at least one air inlet tube close to the atomizing core has an air outlet of the at least one air inlet tube, which is located in a height position inside the cover body lower than the air outlet of the atomizing core.

3. The atomizing assembly according to claim 1, wherein an end of the at least one air inlet tube close to the atomizing core has an air outlet of the at least one air inlet tube, which is located in a height position inside the cover body higher than the air outlet of the atomizing core no more than 20 mm.

4. The atomizing assembly according to claim 1, wherein the at least one air inlet tube is configured to extend vertically inside the cover body.

5. The atomizing assembly according to claim 1, wherein less than or equal to 8 through holes and air inlet tubes are respectively provided, each through hole is in air communication with a respective air inlet tube, and the air inlet tubes are distributed at different positions from each other.

6. The atomizing assembly according to claim 1, wherein the at least one air inlet tube is in form of a circular tube or elliptical tube or polygonal tube.

7. The atomizing assembly according to claim 6, wherein the at least one air inlet tube has a cross-sectional area between 0.15 mm² and 15 mm².

8. The atomizing assembly according to claim 1, wherein an extension length of the at least one air inlet tube inside the cover body is in a range from 5 mm to 200 mm.

9. The atomizing assembly according to claim 1, wherein the at least one air inlet tube is configured to extend outside the cover body, and a baffle is provided below the cover body, with the at least one through hole and an air outlet of at least one the air inlet tube located at an outer side of the baffle and the air outlet of the atomizing core located at an inner side of the baffle.

10. The atomizing assembly according to claim 1, wherein the cover body is provided with a spray tube disposed above the atomizing core.

11. The atomizing assembly according to claim 1, further comprising an airflow driving element, with an exhaust end thereof connected to the at least one through hole at an exterior of the cover body to actively drive the external air into the at least one air inlet tube.

12. A fragrance diffuser, comprising:
a housing;
a high-pressure generation device for providing high-pressure airflow, and
an atomizing assembly provided in the housing, which is configured to produce atomized gas under the high-pressure airflow produced by the high-pressure generation device, the atomizing assembly comprising:
a container for containing liquid to be atomized; and
an atomizer, which comprises a cover body and an atomizing core having an air outlet,
wherein the cover body is provided with at least one through hole, and the atomizer is correspondingly provided with at least one air inlet tube having a certain length in communication with the respective through hole, so as to allow external air to enter the atomizer through the at least one air inlet tube.

13. The fragrance diffuser according to claim 12, wherein an end of the at least one air inlet tube close to the atomizing core has an air outlet of the at least one air inlet tube, which is located in a height position inside the cover body lower than the air outlet of the atomizing core.

14. The fragrance diffuser according to claim 12, wherein an end of the at least one air inlet tube close to the atomizing core has an air outlet of the at least one air inlet tube, which is located in a height position inside the cover body higher than the air outlet of the atomizing core no more than 20 mm.

15. The fragrance diffuser according to claim 12, wherein the at least one air inlet tube is configured to extend vertically inside the cover body.

16. The fragrance diffuser according to claim 12, wherein less than or equal to 8 through holes and air inlet tubes are respectively provided, each through hole is in air communication with a respective air inlet tube, and the air inlet tubes are distributed at different positions from each other.

17. The fragrance diffuser according to claim 12, wherein the at least one air inlet tube is in form of a circular tube or elliptical tube or polygonal tube.

18. The fragrance diffuser according to claim 17, wherein the at least one air inlet tube has a cross-sectional area between 0.15 mm² and 15 mm².

19. The fragrance diffuser according to claim 12, wherein an extension length of the at least one air inlet tube inside the cover body is in a range from 5 mm to 200 mm.

20. The fragrance diffuser according to claim 12, wherein the at least one air inlet tube is configured to extend outside the cover body, and a baffle is provided below the cover body, with the at least one through hole and an air outlet of the at least one air inlet tube located at an outer side of the baffle and the air outlet of the atomizing core located at an inner side of the baffle.
